(19) United States
(12) Patent Application Publication
(10) Pub. No.: US 2002/0192676 A1
(43) Pub. Date: Dec. 19, 2002

(54) Method for determining if a type of bacteria is present in a mixture

(76) Inventors: Angelo J. Madonna, Littleton, CO (US); Francisco Basile, Golden, CO (US); Kent J. Voorhees, Golden, CO (US)

Correspondence Address: CHRISTOPHER J. KULISH, ESQ, HOLLAND & HART LLP, P. O. BOX 8749, DENVER, CO, 80201-8749, US

(21) Appl. No.: 10/063,346

(22) Filed: Apr. 12, 2002

Related U.S. Application Data
(60) Provisional application No. 60/299,033, filed on Jun. 18, 2001.

(51) Int. Cl. C12Q 1/68

(52) U.S. Cl. 435/6

(57) ABSTRACT

The invention is directed to a method for determining whether a particular bacteria is present in a liquid mixture of biological material. In one embodiment, the invention comprises the steps of providing a sampling structure comprised of a body to which antibodies for a particular type of bacteria have been bonded and exposing the sampling structure to a liquid medium that may contain whole cell bacteria for the particular type of bacteria. If such bacteria are present, there is a likelihood that the antibodies will capture some of the bacteria. The method further comprises performing a separation operation to remove materials located adjacent to the sampling structure that may corrupt a subsequent MALDI-TOF analysis, preparing the sampling structure surface with any captured bacteria for MALDI-TOF analysis, subjecting any captured bacteria to a MALDI-TOF analysis to produce a mass spectrum, and analyzing the mass spectrum determine if the particular type of bacteria is present in the liquid mixture.

### CROSS REFERENCE TO RELATED APPLICATIONS

The present application is a continuation application of provisional patent application serial No. 60/299,033, which is incorporated herein by reference.

### FIELD OF THE INVENTION

The present invention is directed to a method for determining if a type of bacteria is resident in a liquid mixture of biological materials.

### BACKGROUND OF THE INVENTION

Presently, there are a number of techniques available for analyzing liquid mixtures of biological material to determine if a particular type of biological material is present. One such technique is enzyme-linked immunosorbent assays (ELISA) in which enzyme-labeled antibodies are used to capture antigens for the antibodies that are present in a mixture. Typically, the enzyme-labeled antibodies fluoresce under appropriate conditions, and this fluorescence is used to determine whether the antigen is present in the mixture. Unfortunately, the enzyme-labeled antibodies are cross-reactive, i.e., susceptible to capturing both target antigens and some non-target antigens. Consequently, if a mixture contains non-target antigens that are captured by the antibodies and there are no target antigens in the mixture, the ELISA method will incorrectly indicate that the target antigen is present,-i.e. provide a false positive. A similar technique, known as radioimmunoassay (RIA), is also susceptible to false positives for similar reasons.

Another technique for determining if a particular type of biological material is present in a mixture involves the use of an affinity capture technique in which a material that is capable of capturing a target biological material is attached to a surface and then used to capture any of the target biological material that may be present in a mixture. Provided there is sufficient exposure, some of the target biological material will be captured or trapped adjacent to the surface. The captured material is then analyzed to determine if the target biological material was present in the mixture. One technique that has been used to analyze the captured material involves extracting proteins from the target biological material and then performing matrix assisted laser desorption/ionization time of flight mass spectrometry (MALDI-TOF/MS) on the extracted proteins. Application of the MALDI-TOF technique produces a mass spectrum that can be analyzed to determine if the proteins for the target biological material are present.

The affinity capture/MALDI-TOF/MS technique has been applied to determine if, for example, a particular type of enzyme or peptide was present in a liquid mixture of biological materials. In this case, the affinity capture technique uses antibodies to capture the target biological material and then uses MALDI-TOF/MS to produce a mass spectrum that can be analyzed to determine if the target biological material was present in the mixture.

Recently, the affinity capture/MALDI-TOF/MS technique has been applied to determine if bacteria are present in a liquid mixture of biological materials. The affinity capture portion of the technique involves exposing lectins that are tethered to a polyethersulfone membranes to trap whole cells of bacteria. The lectins attract bacteria because lectins have a strong affinity for the carbohydrates found at or near the surface of bacteria cells. The membrane and any bacteria trapped by the lectins are subjected to a MALDI-TOF/MS analysis to produce a mass spectrum that can be analyzed to determine if bacteria were present in the mixture. A significant drawback associated with this approach is that a number of different kinds of bacteria have carbohydrates that will be attracted to and trapped by the lectin. Consequently, in environments in which there is significant potential for a number of different bacterial species being present, the mass spectra from the MALDI-TOF/MS analysis becomes increasingly convoluted, making it more difficult to unambiguously determine whether or not a particular type of bacteria are present in the mixture. Stated differently, in mixtures with the potential for having a number of different types of bacteria present, it may not be possible or at least difficult to determine whether a particular type of bacteria is present in the mixture.

### SUMMARY OF THE INVENTION

The present invention is directed to a method for making a determination as to likelihood of the presence of a particular bacteria in a liquid mixture that may contain several different types of biological material. Moreover, the method makes the determination based upon the sampling of whole cell bacteria.

In one embodiment, the method comprises providing an immuno-sampling structure that has an affinity for whole cells of a particular type of bacteria, i.e., a target bacteria. The immuno-sampling structure is comprised of a body with a surface and one or more antibodies for the target bacteria that are attached to the surface. The antibodies operate to attract whole cells of the target bacteria and, provided there is sufficient exposure, capture whole cells of the target bacteria.

The method further comprises exposing the immuno-sampling structure to a liquid medium that is being scrutinized to determine whether any of the target bacteria are present. The method is particularly applicable to situations in which the liquid medium contains or potentially contains several different types of biological material (e.g., blood, sputum, urine, water etc.) because the antibodies are able to selectively capture whole cells of the target bacteria and ignore most of any other biological material that may be present in the liquid medium. If any whole cells of the target bacteria are present in the liquid medium and there is sufficient exposure, the antibodies will attract and capture the cells.

After the exposure of the immuno-sampling structure to the liquid medium, the structure and any whole cells of the bacteria that are attached to the structure via the antibodies are subjected to a separation step to remove undesirable material that may be located adjacent to the structure. The removal of undesirable material improves the signal-to-noise ratio of the data produced by the subsequent MALDI-TOF/MS analysis and thereby facilitates the determination of whether or not the target bacteria is present in the liquid mixture.

After the separation step, the immuno-sampling structure and any associated whole cell bacteria are prepared for MALDI-TOF/MS analysis. Typically, this step comprises applying a MALDI matrix to the immuno-sampling structure and any captured whole cells of the target bacteria. The matrix is an organic acid that serves to break the interactions between the antibodies and any of the captured whole cells of the target bacteria, thereby releasing the whole cells into the matrix. Since only the material that is in the matrix and not attached to the surface will be ionized during the MALDI-TOF/MS process, this separation of the antibodies from any whole cells of the target bacteria significantly reduces the contribution to the mass spectrum produced during the MALDI-TOF/MS process that is attributable to the antibodies. In other words, the spectrum produced by the MALDI-TOF/MS analysis will contain little, if any, signal that is attributable to the antibodies.

Once prepared, the immuno-sampling structure and any associated whole cell bacteria are subjected to a MALDI-TOF/MS analysis that produces mass spectral data. To elaborate, the MALDI process involves directing a laser beam at the sample-matrix to desorb and ionize any bacterial proteins that are present with little fragmentation. Because there is little fragmentation, the resulting ionized bacterial proteins have large masses. The ionized bacterial proteins are applied to a time-of-flight mass spectrometer that produces a mass spectrum that typically includes a number of “peaks,” including peaks for proteins with large masses.

The mass spectrum provides a “fingerprint” that is then analyzed by any one of a number of different techniques to determine if the mass spectrum comprises the spectrum for the target bacteria of concern. Due to the use of the antibodies to capture whole cells of the target bacteria and the separating of the antibodies from other material in the mixture, the sample subjected to the MALDI-TOF/MS analysis is unlikely to be significantly corrupted with non-targeted biological material. As a consequence, the spectrum produced by the MALDI-TOF analysis is likely to have little, if any, “background noise.” Further, while the use of certain antibodies, such as polyclonal antibodies, to capture the target bacteria are susceptible to cross-reactivity (i.e., capturing some non-target bacteria), like the ELISA and RIA methods, the MALDI-TOF analysis provides considerably more data in the form of a number of peaks and peaks associated with high mass proteins. This greater amount of data renders it unlikely that the method will produce a false positive.

The immuno-sampling structure utilized can take any number of forms depending on the particular application. In one embodiment of the method, an immuno-sampling structure is provided in which the body is in the form of magnetic bead. Typically, the bead has a diameter of 0.5-10 μm. At least one but typically multiple antibodies for the target bacteria are attached to the surface of bead. The magnetic characteristic of the bead is used to separate the bead and any associated whole cell bacteria from a substantial portion of the liquid medium. The beads exhibit a large surface area and, consequently, are able to provide large antibody surface densities. A larger antibody surface density improves the signal strength in the subsequent MALDI-TOF analysis and reduces detection limits. Further, such beads are readily circulated in liquid mixtures. As a consequence, the time needed for a sufficient exposure to the liquid medium may be reduced relative to other sampling structures.

In another embodiment, an immuno-sampling structure is provided in which the body is in the form of a conventional MALDI probe (e.g., glass slide, gold plate etc.), which provides a surface that does not react during the application of laser energy to a sample on the probe. By modifying a conventional MALDI probe so that antibodies are attached to the surface of the probe, the probe serves both as the immuno-sampling structure and the substrate for the MALDI process. This dual functionality eliminates the step of transferring an immuno-sampling structure to a MALDI probe that is required in other approaches.

In another embodiment, an immuno-sampling structure is provided that is capable of capturing two or more different whole cell target bacteria and/or capturing whole cell target bacteria from two or more biological liquid mixtures during a single exposure period, which improves throughput in certain situations relative to other immuno-sampling structures.

For example, in the case of testing a single biological mixture for two different types of whole cell target bacteria, the immuno-sampling structure comprises a surface with antibodies for one type of target bacteria attached to one area of the surface and antibodies for the other type of target bacteria attached to a different area on the surface. The immuno-sampling structure is sufficiently exposed to the mixture so as to capture at least some of any of the two types of target bacteria that may be present in the mixture.

In the case of testing two mixtures for one type of whole cell target bacteria, the immuno-sampling structure comprises a surface with antibodies for the target bacteria attached to two distinct locations. The immuno-sampling structure further comprises an “exposure” structure that interfaces with the surface and exposes the first mixture to the antibodies at the first location but not to the antibodies at the second location and exposes the second mixture to the antibodies at the second location but not to the antibodies at the first location at substantially the same time as the first mixture is being exposed.

In the case of testing two mixtures for two different types of whole cell target bacteria, the immuno-sampling structure comprises antibodies for the first target bacteria at two distinct locations and antibodies for the second type of target bacteria at two distinct locations on the surface and the “exposure” structure interfaces with the surface such that the first mixture is exposed to both the first and second antibodies and the second mixture is exposed to both the first and second antibodies but at different locations than the first mixture was exposed to the first and second antibodies.

In another embodiment, the method is adapted to determine if a patient has a particular type of bacterial infection. In such an application, the method comprises a collection step in which a liquid mixture is obtained or produced that is suitable for exposure to the immuno-sampling structure. Blood, urine and sputum are obtained by conventional means and typically do not require any further processing. However, samples that are obtained by swabs (e.g., throat swabs) and the like must be processed to suspend the sampled material in a liquid form.

In yet a further embodiment, the method is adapted to the analysis of bacteria that are in the form of powders (e.g., anthrax). In such applications, the method further comprises a collection step in which a collected powder is suspended in an appropriate liquid solution before the sample is exposed to the immuno-sampling structure.

Another embodiment of the method is adapted to the testing of water. In the testing of water, the desired concentrations of certain bacteria are very low (e.g., one organism per 100 liters). In such applications, while the sample is in a liquid form, the method further comprises a collecting step in which any organisms are subjected to a concentration process before exposure to the immuno-sampling structure. For example, in the case in which the desired concentration is one organism per 100 liters of water, it is typically desirable to obtain a concentrated sample by filtering several hundred liters of water.

### BRIEF DESCRIPTION OF THE DRAWINGS

FIG. 1 illustrates a typical immuno-sampling structure;

FIG. 2 illustrates an immunomagnetic bead sampling structure;

FIG. 3 illustrates an embodiment of a portion of the invention process that involves immunomagnetic bead sampling structures;

FIG. 4 illustrate an immuno-sampling structure suitable for immuno-trapping of a number of different types of bacteria;

FIGS. 5A-5B illustrates a multiple liquid mixture exposure member that is used with the immuno-sampling structure shown in FIG. 4 to expose multiple mixtures to one or multiple antibodies on the structure shown in FIG. 4;

FIGS. 6A-6B respectively show MALDI-TOF mass spectra of Salmonella choleraesuis bacteria that were captured with immunomagnetic beads and of S. choleraesuis bacteria by themselves;

FIGS. 7A-7C respectively show MALDI-TOF mass spectra of sample concentrates of anti-Salmonella immunomagnetic beads removed from suspensions of Staphylococcus aureus suspension, a buffer, and Salmonella boydii;

FIGS. 8A-8B respectively show MALDI-TOF mass spectra acquired from concentrated sample suspensions of anti-Salmonella immunomagnetic beads removed from a bacterial mixtures of S. choleraesuis, S. aureus, and S. boydii and a mixture of S. choleraesuis, Bacillus circulans, and Pseudomonas aeruginosa; and

FIGS. 9A-9C respectively show MALDI-TOF mass spectra from sample concentrates of anti-Salmonella immunomagnetic beads isolated from biological solutions of chicken blood, river water, and urine, each seeded with S. choleraesuis.

### DETAILED DESCRIPTION

The invention is directed to a method for quickly determining, with a relatively high degree of specificity, whether a particular bacteria is present in a liquid mixture of biological materials. Generally the method comprises: (a) providing an immuno-sampling structure with a surface to which antibodies for a particular bacteria of interest, hereinafter referred as a target bacteria, are tethered; (b) exposing the immuno-sampling structure to the mixture that may contain whole cells of the target bacteria; (c) separating the immuno-sampling structure and whole cells of the target bacteria that have been captured from the sampled mixture; (d) preparing any whole cell bacteria captured by the immuno-sampling structure and retained by the immuno-sampling structure subsequent to the separating step for MALDI-TOF analysis; (e) performing the MALDI-TOF analysis to produce a mass spectrum; and (f) determining if the mass spectrum indicates that the target bacteria was in the sampled mixture.

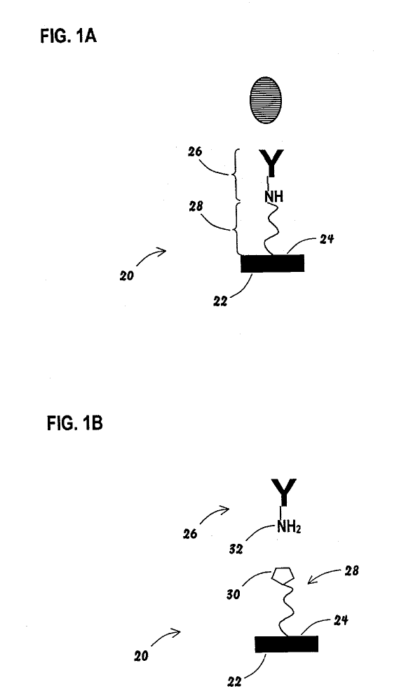

With reference to FIG. 1A, an embodiment of an immuno-sampling structure 20 is described. The structure 20 is comprised of a body 22, a surface 24, and an antibody 26 for the particular bacteria of interest (e.g., E. coli) that is attached to the surface 24. In the illustrated embodiment, the antibody 26 is indirectly attached to the surface 24. To elaborate, and with reference to FIG. 1B, associated with the body 22 is a succinimidylpropionate (SDP) group 28 with a fixed end that is attached to the surface 24 via a siloxane attachment and a free or attachment end with a protein 30. The SDP group 28 is a cross-linking reagent for binding proteins via their amino groups. In this case, the protein 30 of the SDP group 28 binds with a primary amine 32 associated with the antibody 26. The manufacture of the sampling structure 20 is achieved by obtaining a commercially available body 22 with the attached SDP group 28 and then flowing a solution containing the antibody for the target bacteria over the surface 24. The primary amine 32 bonds with the protein 30 to achieve the indirect attachment of the antibody 26 to the surface 24 of the body 22. It should be appreciated that the SDP group 28 is one type of coupling reagent and that other reagents known in the art are also suitable. Further, it is also possible to directly couple an antibody 26 to the surface of a body. The surface 24 can take any number of forms, including, but not limited to, the wall of a container or tube and a surface of a plate.

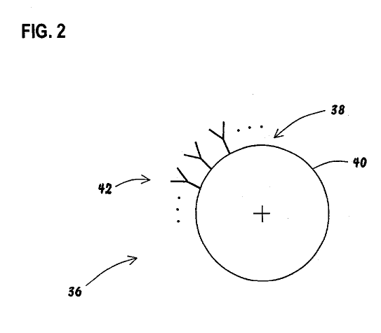

With reference to FIG. 2, one type of immuno-sampling structure that is particularly useful is an immunomagnetic bead 36 that is comprised of a magnetic bead 38 with a surface 40 and a plurality of antibodies 42 for a target bacteria that are attached to the surface 40, directly or indirectly. Typically, the magnetic bead 38 is comprised of a spherical bead (approx. 0.5-100 μm in diameter) of superparamagnetic material, such as Fe₃O₄, that is only magnetic in the presence of a magnetic field and a polymer coating over the surface of the bead. The use of the immunomagnetic bead 36 has a number of advantages. Among these advantages are that: (a) the antibodies in the immunomagnetic bead 36 facilitate the capture of the target bacteria, while de-selecting or ignoring most other biological material that may be present in the sampled mixture; (b) the magnetic characteristic of the immunomagnetic bead 36 facilitates separation of the structure and any captured whole cell bacteria from other biological material in a sampled mixture; (c) the magnetic bead 38 has a large surface area that allows a high density of antibodies 42 to be achieved on the surface 40; (d) the immunomagnetic bead 36 has a small footprint that is smaller than or relatively close in area to the spot size of the laser beam used in many MALDI systems; (e) the high density of antibodies 42 and a footprint for the immunomagnetic bead 36 that is smaller than or relatively close in area to the spot size of the laser beam used in MALDI systems yields a high number of ions for TOF mass spectrometry analysis that, in turn, produces a strong or robust mass spectrum for subsequent analysis; and (f) because the immunomagnetic bead 36 is small and light, it is easily dispersed and circulated in liquid mixtures.

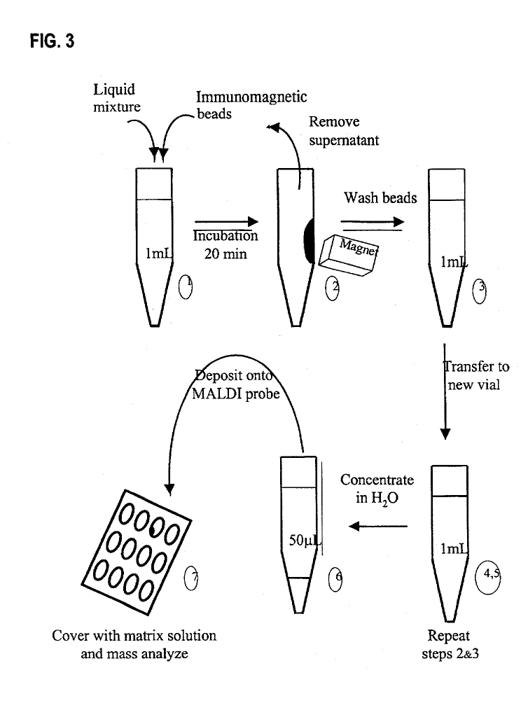

With reference to FIG. 3, an embodiment of the process for determining whether a target bacteria is present in a liquid mixture of biological materials that uses the immunomagnetic bead 36 is described. Initially, a plurality of immunomagnetic beads that each has a plurality of monoclonal and/or polyclonal antibodies for the target bacteria are exposed to the liquid mixture that is being analyzed to determine whether it contains a target bacteria and is likely to contain a number of different types of biological materials by bringing the immunomagnetic beads and the mixture together in a container. The plurality of immunomagnetic beads and the liquid mixture are allowed to incubate for a period of time. Preferably, action is taken during the incubation period to promote circulation of the immunomagnetic beads within the mixture. Typically, this involves either continuously shaking the container or continuously stirring of the mixture, but other actions are also feasible. When circulation of the immunomagnetic beads is promoted by a continuous type of action, twenty minutes has been found to be an adequate incubation period, i.e., a sufficient period of time for at least some of the antibodies to attract and capture enough whole cells of some of any of the target bacteria that may be present in the mixture for MALDI-TOF analysis. If circulation of the beads is not undertaken or discontinuous, a longer incubation period may be required. Further, even if continuous circulation of the beads occurs, a lesser incubation period may be feasible.

Once the incubation period is over, the immunomagnetic beads and any associated whole cells of the target bacteria that have been captured by the antibodies are separated from a substantial portion of the remaining mixture. Initially, this separation is achieved by applying a magnetic field to the container. As a consequence of the paramagnetic material used in the immunomagnetic beads, the application of the magnetic field renders the immunomagnetic beads magnetic. Further, the magnetic field attracts the beads to the interior surface of the container and traps or holds the beads against the interior surface. With the immunomagnetic beads trapped against the interior surface of the container, a substantial portion of the remaining liquid mixture is removed from the container using suction.

At this point, there is still likely to be some material from the liquid mixture adhering to the immunomagnetic beads or located adjacent to the beads that is not a captured target bacteria. Such material may degrade the quality of the mass spectrum that is to be subsequently produced using MALDI-TOF mass spectrometry and make the determination of whether the target bacteria is present in the mixture difficult. Consequently, it is desirable to separate the immunomagnetic beads and any captured whole cells of the target bacteria from such material to improve the quality of the mass spectrum. To separate the beads and any captured whole cells of the target bacteria from such material, the beads are subjected to a washing and preferably several washings. For a washing, the magnetic field is removed and the beads are suspended in an inert liquid that will not damage any whole cells of the target bacteria that have been captured by the antibodies, such as phosphate buffered saline (PBS). Circulation of the beads in the inert liquid is promoted to dislodge any of the liquid mixture adhering or located adjacent to the beads. Typically, this is done by shaking the container, but stirring and other approaches are possible. Presently, twenty seconds of such circulation has been found to be adequate, but less time may be adequate in particular situations and more time needed in other situations. The inert liquid and beads are then transferred to a new container and a magnetic field is then applied to the new container to trap the immunomagnetic beads against the interior of the new container. The inert liquid with any dislodged material is then separated from the beads using suction. In the illustrated embodiment, the washing process is repeated two more times.

It should be appreciated that a number of modifications to the separation of the immunomagnetic beads and any associated whole cells of the target bacteria are possible. For instance, the removal of the remaining liquid mixture or inert liquid can be accomplished by other methods known in the art other than suctioning, such as decanting. Further, instead of removing the remaining liquid mixture or inert liquid from the container, it is also feasible to use the magnetic field to move the immunomagnetic beads out of a container and into another container. In addition, it may not be necessary to transfer the immunomagnetic beads and inert liquid to new containers.

At this point, there should be little, if any, material from the sampled liquid mixture adhering to or located adjacent to the immunomagnetic beads that is not a captured whole cell of the target bacteria. Any such remaining material is likely to be present is such small quantities as to have little effect on the subsequent mass spectrum produced by MALDI-TOF mass spectral analysis. Consequently, the immunomagnetic beads and any captured whole cells of the target bacteria are prepared for the MALDI-TOF analysis by suspending the beads in a small amount of deionized water. The deionized water creates a suspension that facilitates transfer of at least a portion of the beads to a MALDI probe and does not add any material to the beads that would corrupt or inject noise into the subsequently produced mass spectrum. At least a portion of the concentrated bead suspension is transferred to a MALDI sample probe. In the illustrated embodiment, 2 μL aliquots of the concentrated bead suspension are deposited on the MALDI sample probe. The concentrated bead suspension deposited on the probe is allowed to dry (i.e., the water is allowed to evaporate) under ambient conditions. The resulting sample spots are then overlaid with a MALDI matrix solution. The solution comprises an acid that disrupts the hydrogen bonds between the antibodies and any captured whole cells of the target bacteria and thereby allows the “captured” target bacteria to be independent within the matrix. Consequently, during the subsequent MALDI portion of the MALDI-TOF step, proteins of the captured target bacteria and not the antibodies will be ionized and analyzed using TOF mass spectrometry. One suitable MALDI matrix solution includes 12.5 mg of ferulic acid in 1 mL of 17% formic acid, 33% acetonitrile and 50% deionized water (by volume).

Once the matrix has dried, the MALDI-TOF process is applied to the sample-matrix on the MALDI probe. Generally, the MALDI portion of the process involves exposing the sample-matrix with a laser pulse. The matrix absorbs most of the energy from the light pulse and releases the energy in the form of thermal and kinetic energy that is transferred to the molecules in the sample. Further, bacterial proteins are desorbed and ionized with minor fragmentation, thereby preserving the large mass proteins of the bacteria for analysis by a TOF mass spectrometer. The ionized proteins are applied to a TOF mass spectrometer that produces a mass spectrum of the ionized proteins. The mass spectrum produced by the TOF mass spectrometer is compared to the known mass spectrum for the target bacteria to determine if the target bacteria was present in the sampled liquid mixture.

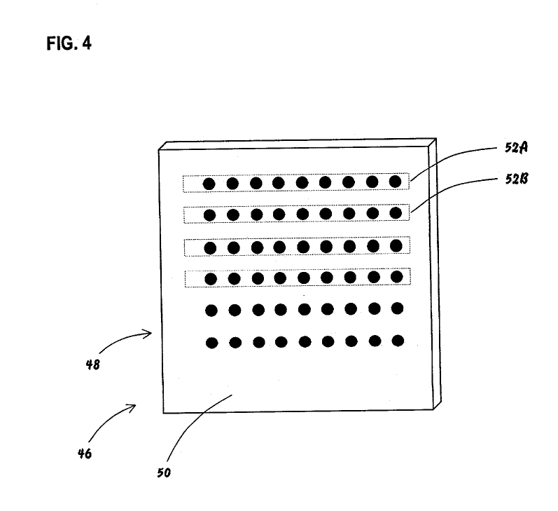

With reference to FIG. 4, an embodiment of an immuno-sampling structure 46 that facilitates the testing of a liquid mixture for the presence of multiple different types of whole cell bacteria is described. The immuno-sampling structure 46 has a body 48 that is a MALDI probe, i.e., a body that is suitable for MALDI analysis. Typically, such probes are made of stainless steel with surfaces of glass, gold, stainless steel etc. In any case, the body 48 has a surface 50. Attached to the surface 50, directly or indirectly, are a first line of antibodies 52A for a first type of bacteria and a second line of antibodies 52B for a second and different type of bacteria. Additional lines of antibodies for yet other types of bacteria are feasible.

In use, the immuno-sampling structure 46 is exposed to a liquid mixture. Typically, the exposure is in a container, but the exposure can also be in-situ, such as in a lake. Further, because the mass of the immuno-sampling structure 46 is fairly large, the structure tends to sink in most applications. Consequently, care must be taken to assure that the lines 52A, 52B are exposed to the liquid mixture and not covered. Further, due to the size and mass of the immuno-sampling structure 46, circulating the structure 46 in the liquid mixture to promote the capture of any whole cell bacteria in the mixture by the antibodies may not be feasible. In such situations, if feasible, the liquid mixture should be stirred or otherwise caused to circulate past the immuno-sampling structure 46. If circulation of the immuno-sampling structure 46 and/or the liquid mixture is not possible, exposure time may need to be increased.

Following exposure of the immuno-sampling structure 46 to the liquid mixture, the liquid mixture and structure 46 with any captured whole cell bacteria are separated from one another. The immuno-sampling structure 46 is then subjected to one or more washings with an inert solution to dislodge material from the liquid mixture that is located adjacent to the structure 46.

After the washing or washings, immuno-sampling structure 46 and any whole cell bacteria that have been captured are prepared for the MALDI portion of the MALDI-TOF step by being allowed to dry. Once dry, a MALDI matrix solution is applied to the first line 52A of antibodies and the second line 52B of antibodies.

After the solution has dried, the first line 52A of antibodies and second line 52B of antibodies are subjected to the MALDI-TOF mass spectrometry process. The first line 52A of antibodies and second line 52B of antibodies 52B are sequentially subjected to the MALDI-TOF mass spectrometry process if only one MALDI-TOF mass spectrometer machine is available. If, however, two MALDI-TOF mass spectrometer machines are available, parallel analysis of the first and second lines 52A, 52B is feasible. In either case, the mass spectrums produced for the first and second lines 52A, 52B are analyzed to determine whether there is a match with the first type of target bacteria and/or the second type of target bacteria.

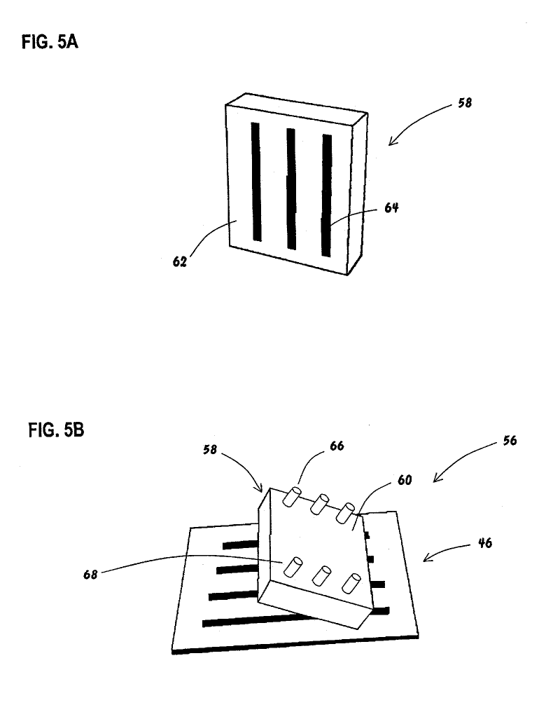

With reference to FIGS. 5A-5B, an embodiment of an immuno-sampling structure 56 that facilitates the testing of more than one liquid mixture to determine if one or more different types of bacteria are present in each mixture is described. The immuno-sampling structure 56 comprises the immuno-sampling structure 46 (FIG. 4) with one line of antibodies for one type of target bacteria or multiple lines of antibodies with each line for a different target bacteria. The immuno-sampling structure 56 further comprises a multiple liquid mixture exposure member 58 that facilitates the simultaneous exposure of multiple mixtures to one or more lines of antibodies while preventing the mixtures from commingling with another. The member 58 has a top side 60 and bottom side 62 that interfaces with the surface 50 of the of the structure 46. The member 58 further comprises one but more typically multiple channels 64 that each have an inlet port 66 and an outlet port 68 and are open on the bottom surface 62. The bottom side 62 comprises rubber or some other material that is capable of making a seal against the surface 50 such that, when the member 58 is sealed against the surface 50, the liquid mixtures in the channels 64 do not intermingle with one another on the surface 50 but are exposed to antibodies. A possible modification to the member 58 is to have a single port that is used to inject a mixture and remove the mixture from a channel.

Operation of the immuno-sampling structure 56 begins with establishing a seal between the member 58 and the surface 50 and orienting the member 58 such that the open side of each of the channels 64 that is to carry a liquid mixture intersects each of the lines of antibodies that have been established on the surface 50. Exposure of the mixtures to the line or lines of antibodies involves circulating each mixture through one of the channels 64 via the inlet port 66 and outlet port 68 for the channel. Following exposure, the member 58 is separated from the surface 50 and the immuno-sampling structure 46 is processed as previously described.

### EXAMPLE

Bacteria Growth

Stock cultures of Salmonella choleraesuis (ATCC 14028), Shigella boydii (ATCC 9207), Pseudomonas aeruginosa (ATCC 25619), Staphylococcus aureus (ATCC 12600), and Bacillus circulans (ATCC 61) were purchased from the American Type Culture Collection (ATCC, Manassas, Va.). Salmonella choleraesuis and S. boydii are both Biosafety Level 2 organisms and should be handled with extreme care. Consequently, all microbiological procedures were preformed in a certified Biosafety level 2 facility using proper sterilization procedures.

Bacteria were grown in trypticase soy broth (Difco, Detroit, Mich.) with incubation at 37° C. and enumerated using a Petroff-Hauser counting chamber (Hausser Scientific, Horsham, Pa.). For direct MALDI analysis of the stock cultures, the cells were separated from the growing media by centrifugation (5000 g for 3 min) and washed twice with PBS. The cellular pellet was then reconstituted into deionized water and deposited directly onto the MALDI sample probe in 2 μL aliquots.

Sample Preparation and Immunomagnetic Separation.

Bacteria suspensions were prepared in 1.5 mL microcentrifuge tubes (Brinkmann Instruments, Inc., Westbury, N.Y.) by combining 100 μL of broth media with 900 μL of phosphate buffer saline (PBS, 0.01 M Na₂HPO₄, 0.15M NaCl titrated to pH 7.35 with HCl). Bacterial mixtures were made by adding 100 μL of three different broth suspensions to 700 μL of buffer. Spiked biological samples of chicken blood, river water, and human urine were prepared by spinning down a broth suspension of S. choleraesuis (centrifugation at 4000G for 3 min) and resuspending the bacterial pellet in 1 mL of each of the respective liquids. For sensitivity studies, samples were serially diluted with PBS.

Dynal anti-Salmonella Dynabeads® (Lake Success, N.Y.) were supplied in a suspension of PBS (pH 7.4) with 1.0% bovine serum albumin and 0.02% sodium azide. The IMS procedure used in this investigation is illustrated in FIG. 3. In the first step, the immunomagnetic beads were added in 30 μL aliquots to the bacteria sample suspensions and incubated for 20 min at room temperature with continuous shaking. The second step involved concentrating the beads to the side of the sample tube using a magnetic particle concentrator (Dynal) and suctioning off the supernatant using a 1 mL pipette. In the third step, the magnet was removed and the beads were resuspended in 1 mL of fresh PBS with vigorous shaking for 20 sec to wash away any nonspecifically adhering components. The bead suspension was then transferred to a new tube before moving on to the next step. In the fourth and fifth steps, the beads were rewashed by repeating the previous two steps. The sixth step was executed by using the magnetic particle concentrator to remove the buffer and concentrate the beads into 50 μL of deionized water. In the seventh and final step, 2 μL aliquots of the concentrated bead suspension were deposited onto the MALDI sample probe using a micropipette.

MALDI-TOF Mass Spectrometry.

The sample spots delivered to the MALDI probe were dried under ambient conditions prior to being overlaid with 1 μL of matrix solution (12.5 mg of ferulic acid in 1 mL of 17% formic acid: 33% acetonitrile: 50% deionized H₂O). All subsequent mass spectra were generated on a Voyager-DE STR (AB Biosystems, Framingham, Mass.) MALDI-TOF mass spectrometer, operating in the positive linear mode. The following parameters were used: accelerating voltage 25 kV, grid voltage 70% of accelerating voltage, extraction delay time of 65 nsec, and low mass ion gate set to 4 kDa. The laser intensity (N₂, 337 nm) was set just above the ion generation threshold and pulsed every 300 ns. Mass spectra were acquired by accumulating 100 laser shots from five different sample spots with the 500 shots averaged to give one spectrum. The raw intensity spectra were smoothed (three standard deviation noise removal) and calibrated externally using the molecular ions for insulin B, thioredoxin, apomyoglobin (AB Biosystems).

Results.

Magnetic beads coated with polycolonal antibodies raised against all serotypes of Salmonella were used to isolate and concentrate S. choleraesuis from an aqueous solution using the procedure illustrated in FIG. 3. The resulting suspension of concentrated immunomagnetic beads and captured bacteria were deposited directly onto a MALDI sample probe in preparation for mass analysis. Each sample was subsequently treated with a ferulic acid matrix solution that was developed to produce high molecular weight protein signals from whole cellular bacteria. It is believed that during solvation, the matrix solution disrupts the cells to a limited degree causing the release of weakly bound membrane and cellular wall proteins. These liberated proteins then become embedded within the crystal as it forms and are eventually ionized and detected following laser irradiation.

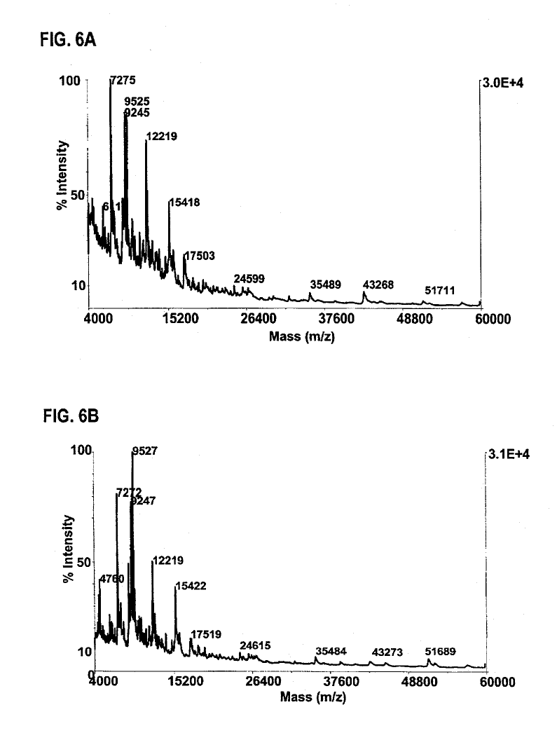

The resulting MALDI-TOF mass spectrum of the S. choleraesuis sample prepared using the immuno-magnetic separation (IMS) procedure is shown in FIG. 6A. For comparison, a sample consisting only of S. choleraesuis (no magnetic beads) was also prepared and mass analyzed under the same conditions (FIG. 6B). Both spectra display the same protein peaks indicating that the presence of the immunomagnetic beads did not produce a significant background signal or alter the resulting mass spectrum produced by S. choleraesuis. The entire procedure (isolation and detection) was accomplished in less than 1 hr demonstrating the feasibility of using IMS-MALDI as a rapid method for detecting bacteria from contaminated fluid samples.

The concentration of the bacterial suspension used to produce the mass spectrum shown in FIGS. 6A, 6B was determined to be ˜3.2×10⁸ cells/mL by a cell counter. Using serially diluted samples of S. choleraesuis in buffer, the detection limit for the IMS-MALDI procedure was found to be ˜1.0×10⁷ cells/mL. This value compares favorably to the lectin affinity capture study where bacterial suspensions of 1.0×10⁹ cells/mL were used. Furthermore, the incubation time used was held to 20 min, while the lectin capture technique required 2 hr for optimal binding to occur.

The signature pattern of proteinaceous spectral peaks provided by MALDI-TOF offers the analyst a high level of specificity for identifying intact microorganisms. This prominent feature becomes particularly important when mass spectrometry is utilized as the end-point detector for immunoassays, which are notorious for giving false-positive results due to cross-reactions between antibodies and nonspecific organisms. Under these circumstances, the production of any signals other than those expected from the targeted bacterium would be interpreted as belonging to a cross-reacted species and appropriately reported as a negative test. In the mass spectrum shown in FIGS. 6A and 6B, several mass peaks distinct to S. choleraesuis were observed. The mass peak at 7275 Da. was previously identified as the cold-shock-like protein C and has been detected for several different members of the family Enterobacteriaceae. In addition, the peak detected at 9.525 Da. has also been recognized as a family-specific biomarker for Enterobacteriaceae while the mass peak at 9245 Da. was designated as genus specific biomarker for species of Salmonella. These specific biomarkers, in conjunction with the higher mass peaks at 12.2 kDa., 14.4 kDa., 17.5 kDa., 35.5 kDa., 43.3 kDa., and 51.7 kDa. should facilitate the explicit identification of S. choleraesuis. However, the last two mass peaks, 43.3 kDa. and 51.7 kDa., were not detected at appreciable intensities for cultures grown for less then 24 hrs.

Another area of concern when utilizing immunomagnetic beads is the problem of carry-over due to physical adsorption of nonspecific components with the surfaces of the beads and microcentrifuge tubes. Preliminary work with stock cultures of Staphylococcus aereus incubated with the anti-Salmonella immunomagnetic beads showed that considerable carry-over was occurring. Evidence of this undesirable effect was provided by the appearance of S. aereus protein peaks in the MALDI-TOF mass spectra obtained from the recovered anti-Salmonella beads. It has previously been shown that the amount of carry-over can be reduced significantly by transferring the immunomagnetic beads to a new tubes following each washing step. This added measure compensates for the large number of nonspecific components that strongly absorbed to the inside surfaces of the tubes. During this investigation, it was determined that two successive wash steps followed each time by transferring the bead suspension to new tubes was sufficient to eliminated contributions from nonspecific bacteria to the MALDI mass spectra.

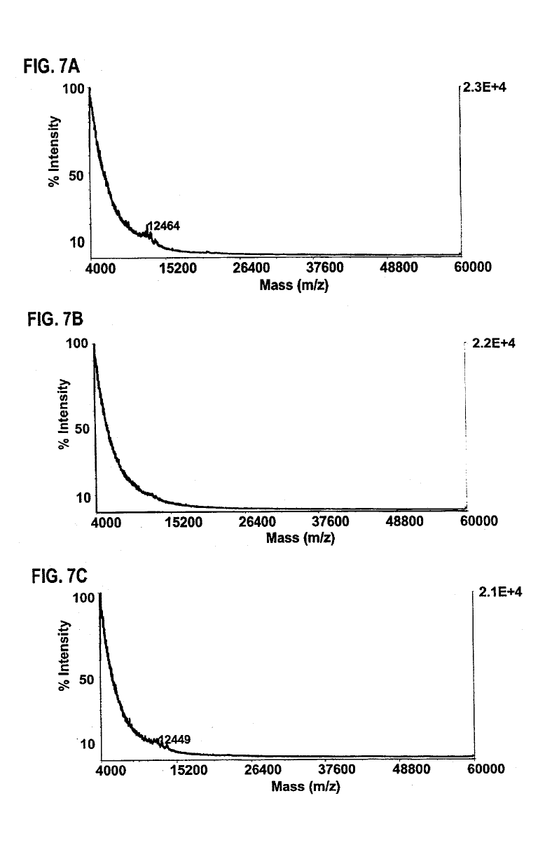

The MALDI-TOF mass spectrum produced from anti-Salmonella immunomagnetic beads recovered from a solution of S. aereus (˜3.0×10⁸ cells/mL) is shown in FIG. 7A. This spectrum is essentially the same as the one produced from just the immunomagnetic beads (FIG. 7B). Except for a minor perturbation around 12 kDa. attributed to the magnetic beads, no other signal above the baseline noise was detected. From the two spectra, it is obvious that S. aereus was successfully removed from the bead suspension during the IMS procedure.

Similar results were obtained when the anti-Salmonella beads were incubated with a suspension of Shigella boydii. The MALDI-TOF mass spectrum shown in FIG. 7C. is from anti-Salmonella beads retrieved from a solution consisting of ˜4.0×10⁸ cells/mL of S. boydii. It is obvious from the spectrum, that the Shigella species was not retained by the immunomagnetic beads. The two genera of Salmonella and Shigella have similar physical characteristics and therefore it is assumed that the beads are binding to the S. choleraesuis cells by specific antigen/antibody interactions. Otherwise, if the trapping of S. choleraesuis was due only to adsorption, then the Shigella species would have been expected to behave in a similar fashion and also produce a protein spectrum when analyzed.

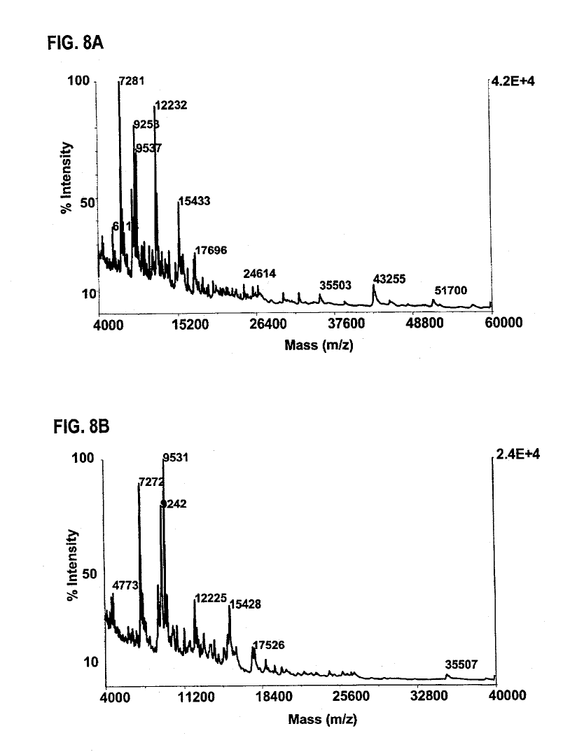

A mixture of three different species of bacteria consisting of approximately the same concentrations (˜1.0×10⁸ cells/mL) of S. choleraesuis, S. aereus, and S. boydii was made and subjected to the IMS-MALDI procedure. The resulting mass spectrum is shown in FIG. 8A. The pattern of protein mass peaks observed in this mass spectrum show that the applied procedure successfully separated and detected S. choleraesuis in the presence of the other two bacteria. This experiment was repeated using a second mixture composed of S. choleraesuis, B. circulans, and P. aeruginosa. The resulting mass spectrum is shown in FIG. 8B. Again, the only protein signals produced were those attributed to the Salmonella species. The second mixture was prepared from cells grown overnight (14 hr), whereas the bacteria in the first mixture were incubated for 2 days. The younger S. choleraesuis cells did not produce any signals above 40 kDa. Consequently, the mass range above this m/z value was not scanned during analysis of the second mixture.

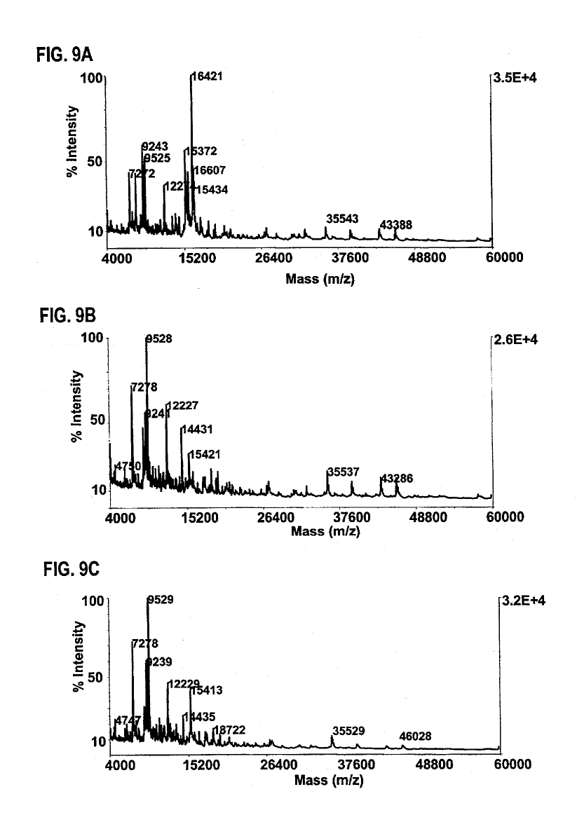

The final objective of this investigation was to evaluate the IMS-MALDI procedure when executed with actual biological solutions. Diverse samples including chicken blood, river water, and human urine spiked to a concentration of ˜1.0×10⁹ cells/mL with the target organism were used. The mass spectra collected from the three complex solutions are shown in FIGS. 9A-9C. In each spectrum, a fingerprint of protein peaks characteristic of S. choleraesuis was observed. The spectra from the river water (FIG. 9B) and urine (FIG. 9C) samples are nearly identical demonstrating the capability of using this technique to analyzing a wide range of different sample types. Several mass spectral peaks were detected in the chicken blood sample (FIG. 9A) that could not be attributed to S. choleraesuis including the peak at 16421 Da., its M ion at 8210 Da., and the ion at 15372 Da. These peaks are suspect of being contributed from either a different Salmonella species that was present in the neat sample, or from another nonspecific component that cross-reacted with the immunomagnetic beads.

The embodiment of the invention described hereinabove is intended to describe the best mode known of practicing the invention and to enable others skilled in the art to utilize the invention.

1. A method for determining whether a particular type of bacteria is present in a liquid mixture comprising:
providing an immuno-sampling structure comprising a body with a surface and antibodies for one type of bacteria that are each attached to said surface;
exposing said immuno-sampling structure to a liquid mixture that may contain whole cells of said one type of bacteria to provide each of said antibodies with an opportunity to capture a whole cell of said one type of bacteria that may be present in said liquid mixture;
separating, after said step of exposing, said immuno-sampling structure and at least some of however many whole cells of said one type of bacteria that have been captured by said antibodies from material in said liquid mixture that may be located adjacent to said sampling structure but that is not a whole cell of said one type of bacteria;
preparing, after said step of separating, said sampling structure and at least some of however many whole cells of said one type of bacteria that are associated with at least a portion of said immuno-sampling structure for MALDI-TOF/MS analysis; and
performing, after said step of preparing, MALDI-TOF/MS analysis on at least some of however many whole cells of said one type of bacteria that were captured by said immuno-sampling structure and prepared for MALDI-TOF/MS analysis to produce a mass spectrum; and
analyzing said mass spectrum to determine if said one type of bacteria was present in said liquid mixture.

2. A method, as claimed in claim 1, wherein:
said body with a surface comprises a bead.

3. A method, as claimed in claim 1, wherein:
said body with a surface comprises a magnetic bead.

4. A method, as claimed in claim 3, wherein:
said magnetic bead is a paramagnetic bead.

5. A method, as claimed in claim 1, wherein:
said body with a surface comprises at least a part of a MALDI probe.

6. A method, as claimed in claim 1, wherein:
said antibodies for one type of bacteria extend over a first area of said surface; and
antibodies for another type of bacteria extend over a second area of said surface that does not overlap with said first area of said surface.

7. A method, as claimed in claim 6, wherein:
said first area has a first portion and a second portion that is separate from said first portion;
said second area has third portion and a fourth portion that is separate from said third portion;
said immuno-sampling structure further comprises an exposure structure for interfacing with said surface so as to present said first and third portions but not said second and fourth portions with a first mixture and so as to present said second and fourth portions but not said first and third portions with a second mixture.

8. A method, as claimed in claim 1, wherein:
said antibodies for said one type of bacteria extend over a first area of said surface and over a second area of said surface that is separate from said first area; and
said immuno-sampling structure further comprises an exposure structure for interfacing with said surface so as to present said first area but not said second area with a first mixture and so as to present said second area but not said first area with a second mixture.

9. A method, as claimed in claim 1, wherein:
said step of separating comprises using a magnetic field.

10. A method, as claimed in claim 1, wherein:
said step of separating comprises washing said immuno-sampling structure.

11. A method, as claimed in claim 1, wherein:
said step of separating comprises washing said immuno-sampling structure multiple times.

12. A method, as claimed in claim 1, wherein:
said step of preparing comprises applying a solution of MALDI matrix to said immuno-sampling structure.

13. A method for determining whether a particular type of bacteria is present in a liquid mixture comprising:
providing a plurality of immunomagnetic beads that each have a surface and antibodies for one type of bacteria that are each attached to said surface;
exposing said plurality of immunomagnetic beads to a liquid mixture that may contain whole cells of said one type of bacteria to provide each of said antibodies with an opportunity to capture a whole cell of said one type of bacteria that may be present in said liquid mixture;
separating, after said step of exposing, at least some of said plurality of immunomagnetic beads and however many whole cells of said one type of bacteria that have been captured by said antibodies associated with said plurality of immunomagnetic beads from material in said liquid mixture that may be located adjacent to said plurality of immunomagnetic beads but that is not a whole cell of said one type of bacteria;
preparing, after said step of separating, said plurality of immunomagnetic beads and however many whole cells of said one type of bacteria that are associated with said plurality of immunomagnetic beads for MALDI-TOF/MS analysis; and
performing, after said step of preparing, MALDI-TOF/MS analysis on whole cells of said one type of bacteria that were captured by said plurality of immunomagnetic beads and prepared for MALDI-TOF/MS analysis to produce a mass spectrum; and
analyzing said mass spectrum to determine if said one type of bacteria was present in said liquid mixture.

14. A method, as claimed in claim 13, wherein:
said plurality of immunomagnetic beads comprises paramagnetic beads.

15. A method, as claimed in claim 13, wherein said step of separating comprises:
using a magnetic field to attract said immunomagnetic beads to a surface; and
establishing relative movement between said immunomagnetic beads that have been attracted to said surface and said liquid mixture to separate said immunomagnetic beads and said liquid mixture.

16. A method, as claimed in claim 15, wherein said step of establishing comprises:
decanting said liquid mixture.

17. A method, as claimed in claim 15, wherein said step of separating comprises:
washing, after said step of establishing, said immunomagnetic beads that have been separated from said liquid mixture with a washing liquid.

18. A method, as claimed in claim 15, wherein said step of separating comprises:
washing, after said step of establishing, said immunomagnetic beads that have been separated from said liquid mixture with a washing liquid; and
establishing relative movement between said immunomagnetic beads that have been washed and said washing liquid to separate said immunomagnetic beads and said washing liquid.

19. A method, as claimed in claim 18, wherein said step of establishing comprises:
decanting said liquid mixture.

20. A method, as claimed in claim 18, wherein said step of separating comprises:
repeating said steps of washing and establishing a multiple times.

21. A method, as claimed in claim 13, wherein:
said step of separating comprises washing said sampling structure multiple times.

22. A method, as claimed in claim 13, wherein said step of preparing comprises:
placing at least a portion of said plurality of immunomagnetic beads on a MALDI probe.

23. A method, as claimed in claim 22, wherein said step of preparing comprises:
applying, after said step of placing, a solution of MALDI matrix to said portion of said plurality of immunomagnetic beads on said MALDI probe.

24. A method for determining whether a particular type of bacteria is present in a liquid mixture comprising:
providing a immuno-sampling structure comprising a body with a surface, first antibodies for a first type of bacteria that are each attached to and extend over a first area of said surface, and second antibodies for a second type of bacteria that are each attached to and extend over a second area of said surface;
exposing said immuno-sampling structure to a liquid mixture that may contain whole cells of said first and second types of bacteria to provide said first antibodies with an opportunity to capture a whole cell of said first type of bacteria that may be present in said liquid mixture and said second antibodies with an opportunity to capture a whole cell of said second type of bacteria that may be present in said liquid mixture;
separating, after said step of exposing, said immuno-sampling structure and however many of said whole cells of said first and second types of bacteria that have been captured by said first and second antibodies from material in said liquid mixture that may be located adjacent to said immuno-sampling structure but that is not a whole cell of either said first or second type of bacteria;
preparing, after said step of separating, said immuno-sampling structure and however many of said whole cells of said first and second types of bacteria that are associated with at least a portion of said immuno-sampling structure for MALDI-TOF/MS analysis; and
performing, after said step of preparing, MALDI-TOF/MS analysis on however many of said whole cells of said first and second types of bacteria that were captured by said immuno-sampling structure and prepared for MALDI-TOF/MS analysis to produce a mass spectrum; and
analyzing said mass spectrum to determine if either said first or second type of bacteria was present in said liquid mixture.

25. A method, as claimed in claim 24, wherein:
said first area has a first portion and a second portion that is separate from said first portion;
said second area has third portion and a fourth portion that is separate from said third portion;
said immuno-sampling structure further comprises an exposure structure for interfacing with said surface so as to present said first and third portions but not said second and fourth portions with a first mixture and so as to present said second and fourth portions but not said first and third portions with a second mixture.
